(19) United States
(12) Patent Application Publication
(10) Pub. No.: US 2009/0088642 A1
(43) Pub. Date: Apr. 2, 2009

(54) ULTRASONIC IMAGING APPARATUS AND ULTRASONIC IMAGING METHOD

(75) Inventor: Tomoo SATO, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM CORPORATION, Tokyo (JP)

(21) Appl. No.: 12/237,589

(22) Filed: Sep. 25, 2008

(30) Foreign Application Priority Data
Sep. 28, 2007 (JP) 2007-255999

(51) Int. Cl. A61B 8/14

(52) U.S. Cl. 600/459

(57) ABSTRACT

In an ultrasonic imaging apparatus, image quality is improved without increase in the number of ultrasonic transducers. The ultrasonic imaging apparatus includes: a transmitting unit for supplying drive signals to plural ultrasonic transducers so as to form an ultrasonic beam having a beam width that covers adjacent two or more transmission lines; a receiving unit for performing phase-matching processing and addition processing on the reception signals to generate RF data in which focuses of ultrasonic echoes are narrowed in positions corresponding to respective pixels of an ultrasonic image to be displayed; an RF data storage unit for storing the RF data with respect to a plural number of transmissions of the ultrasonic beams; and an image signal generating unit for averaging the RF data for the plural number of transmissions and generating an image signal representing the ultrasonic image based on the averaged RF data.

### BACKGROUND OF THE INVENTION

1. Field of the Invention

The present invention relates to an ultrasonic imaging apparatus and an ultrasonic imaging method for acquiring ultrasonic images of an object to be inspected by transmitting and receiving ultrasonic waves to scan the object, and specifically, to an ultrasonic imaging apparatus and an ultrasonic imaging method for acquiring ultrasonic images of the object by using an ultrasonic endoscope for intracavitary scan of the object.

2. Description of a Related Art

In medical fields, various imaging technologies have been developed in order to observe the interior of an object to be inspected and make diagnoses. Especially, ultrasonic imaging for acquiring interior information of the object by transmitting and receiving ultrasonic waves enables image observation in real time and provides no exposure to radiation unlike other medical image technologies such as X-ray photography or RI (radio isotope) scintillation camera. Accordingly, ultrasonic imaging is utilized as an imaging technology at a high level of safety in a wide range of departments including not only the fetal diagnosis in the obstetrics, but also gynecology, circulatory system, digestive system, and so on.

The ultrasonic imaging is an image generation technology utilizing the nature of ultrasonic waves that the ultrasonic waves are reflected at a boundary between regions with different acoustic impedances (e.g., a boundary between structures). Typically, an ultrasonic imaging apparatus (or also referred to as an ultrasonic diagnostic apparatus or ultrasonic observation apparatus) is provided with an ultrasonic probe to be used in contact with the object or ultrasonic probe to be used by being inserted into a body cavity of the object. Alternatively, an ultrasonic endoscope may be used in which an endoscope for optically observing the interior of the object is combined with an ultrasonic probe for intracavity.

In a typical ultrasonic probe, a vibrator (piezoelectric vibrator) having electrodes formed on both sides of a material having a piezoelectric property (a piezoelectric material) is used as an ultrasonic transducer for transmitting and receiving ultrasonic waves. When a pulsed or continuous wave voltage is applied to the electrodes of the vibrator, the piezoelectric material expands and contracts, and generates pulsed or continuous wave ultrasonic waves. Further, the vibrator expands and contracts by receiving propagating ultrasonic waves and generates an electric signal. This electric signal is used as a reception signal of the ultrasonic waves.

Plural ultrasonic transducers are one-dimensionally or two-dimensionally arranged and sequentially driven. The ultrasonic waves transmitted from the respective ultrasonic transducers are synthesized to form an ultrasonic beam for electronic scanning of the object. In the scanning of the object, reception signals obtained by receiving ultrasonic echoes are phase-matched and added, and typically, a sound ray signal is formed in which focuses of the ultrasonic echoes are narrowed to sampling points along a sound ray. Further, the sound ray signal is converted by a digital scan converter (DSC) into an image signal that follows the normal scan system of television signals. In this regard, there are few correspondences between the locations of the sampling points in the sound ray signal and the locations of pixels in the image signal, and accordingly, interpolation processing is performed on the image signal. That contributes to deterioration of image quality.

There are various types of ultrasonic probe depending on the arrangement of ultrasonic transducers such as a linear-array probe, convex-array probe, and annular-array probe, and further, there are various kinds of scan system such as a linear-scan system, sector-scan system, convex-scan system, and radial-scan system.

As an intracavity ultrasonic probe to be used in an ultrasonic endoscope, there is an FNA probe for fine needle aspiration (FNA), which probe is considered as a kind of convex-array probe, for acquiring a part of a living body tissue by inserting a needle into the object while observing an ultrasonic image, or a radial probe for scanning the object according to an electronic radial-scan system. In those ultrasonic probes, scanning of the object is performed by shifting a channel group of ultrasonic transducers to be used to move a transmission beam. Accordingly, in order to acquire accurate ultrasonic images, it is conceivable that a large number of ultrasonic transducers are closely arranged for a narrow movement pitch of the transmission beam.

However, for example, in order to transmit an ultrasonic beam with a pitch of a scan angle of 1°, 80 to 150 channels of ultrasonic transducers are necessary in the FNA probe, and 200 to 360 channels of ultrasonic transducers are necessary in the radial probe. Especially, since 200 or more channels of ultrasonic transducers are necessary in the radial probe, manufacturing of ultrasonic transducers becomes difficult, and the number of coaxial cables for connecting the respective ultrasonic transducers to an ultrasonic imaging apparatus main body is increased and the diameter of the endoscope becomes larger.

Further, when the number of channels of ultrasonic transducers is increased, the number of pins to be used for connecting the ultrasonic probe to the ultrasonic imaging apparatus main body is increased, and a large connector should be used. However, if a large connector is used, the endoscope becomes too large to be inserted into a washing machine for cleaning. On the other hand, it is conceivable that part of connection of ground wires is omitted for connecting a large number of ultrasonic transducers to a connector with a small number of pins. In this case, however, changes in capacitance and so on in the coaxial cables and the connector may vary signal waveforms. From these points of view, it is undesirable to increase the number of channels of ultrasonic transducers.

As a related technology, Japanese Patent Application Publication JP-A-5-92002 discloses an intracavity ultrasonic observation apparatus intended to obtain stable images by reducing the number of signal wires so that multi-element arrayed vibrators are easily incorporated into the limited space within an intracavity probe, and electronically scanning the multi-element arrayed vibrators. The intracavity ultrasonic observation apparatus includes a vibrator main body formed by arranging a large number of vibrator elements in an array and a multiplexer for sequentially selecting the vibrator elements provided within an insertion end part of the intracavity probe, a reception amplifier unit for inputting and amplifying outputs of the vibrator elements selected by the multiplexer via the signal wires extended through the intracavity probe, an A/D converter for converting the signals into digital signals, wave front memories for storing the digital signals correspondingly to the respective vibrator elements as wave front data, and a wave front synthesizer circuit for performing addition processing based on the outputs of the respective wave front memories.

Japanese Patent Application Publication JP-A-7-148167 discloses an ultrasonic diagnostic apparatus intended to obtain ultrasonic tomographic images at a large frame rate with good image quality. The ultrasonic diagnostic apparatus includes a vibrator array formed by arranging plural vibrator elements, a multiplexer for sequentially selecting plural vibrator elements or plural vibrator element groups in parallel for transmitting and receiving ultrasonic waves from the vibrator array, plural transmission circuits for driving the respective vibrator elements or vibrator element groups for transmitting ultrasonic waves from the selected plural vibrator elements or plural vibrator element groups, plural reception synthesizing means for processing reception signals of the ultrasonic waves received by the respective vibrator elements or vibrator element groups to obtain reception signals in respectively corresponding directions, and a digital scan converter for creating one ultrasonic image based on the reception signals from these plural reception synthesizing means.

Japanese Patent JP-B-3408284 discloses an acoustic imaging apparatus intended to improve the resolving power without reducing the frame rate of a system. In the acoustic imaging apparatus, the order of signal processing is changed in order to improve the resolving power of images using signal phase information, which is normally lost in the reconfiguration process. That is, the generated signals are detected by a converter and scan-conversion or data interpolation is performed on the signals before processing is performed with limitation.

Japanese Patent Application Publication JP-P2006-87668A discloses an ultrasonic imaging apparatus by which ultrasonic images with improved image quality can be obtained by effectively utilizing ultrasonic echo information that is obtained by transmission and reception of ultrasonic waves. The ultrasonic imaging apparatus includes an ultrasonic probe including plural ultrasonic transducers, transmitting means for respectively supplying drive signals to the plural ultrasonic transducers for scanning of the object, scan system converting means for generating waveform signals representing waveform information of ultrasonic echoes in the respective pixels to be displayed by performing phase-matching processing on reception signals, image signal generating means for generating an image signal based on the waveform signals, storage means for respectively storing plural kinds of image signals with respect to plural different transmission directions, and image synthesizing means for obtaining one kind of image signals by synthesizing images representing by the plural kinds of image signals.

According to JP-A-5-92002 and JP-A-7-148167, although the number of channels of ultrasonic transducers is not reduced, the number of cables is reduced by providing the multiplexer for sequentially selecting the ultrasonic transducers in the ultrasonic probe. However, it is practically difficult to provide such a multiplexer in an intracavity ultrasonic probe to be used in an ultrasonic endoscope. Further, JP-B-3408284 and JP-P2006-87668A disclose that the image quality is improved without increase in the number of channels of ultrasonic transducers. However, there is still room for improvement of image quality.

### SUMMARY OF THE INVENTION

The present invention has been achieved in view of the above-mentioned problems. A purpose of the present invention is, in the case where an ultrasonic probe is provided in a limited internal space as in an ultrasonic endoscope, for example, to provide an ultrasonic imaging apparatus and an ultrasonic imaging method by which image quality can be improved without increasing the number of channels of ultrasonic transducers, alternatively, the number of channels of ultrasonic transducers can be reduced while good image quality is maintained.

In order to accomplish the purpose, an ultrasonic imaging apparatus according to the present invention includes: an ultrasonic probe including plural ultrasonic transducers for forming an ultrasonic beam according to drive signals to transmit the ultrasonic beam toward an object to be inspected, and receiving ultrasonic echoes generated within the object to generate reception signals; transmitting means for supplying, when sequentially transmitting ultrasonic beams along plural different transmission lines from the ultrasonic probe, drive signals to the plural ultrasonic transducers so as to form an ultrasonic beam having a beam width that covers adjacent two or more transmission lines; receiving means for performing phase-matching processing and addition processing on the reception signals obtained by the plural ultrasonic transducers receiving the ultrasonic echoes to generate RF (radio frequency) data in which focuses of ultrasonic echoes are narrowed in positions corresponding to respective pixels of an ultrasonic image to be displayed; an RF data storage unit for storing the RF data generated by the receiving means with respect to a plural number of transmissions of the ultrasonic beams; image signal generating means for loading the RF data from the RF data storage unit, averaging the RF data for the plural number of transmissions of the ultrasonic beams, and generating an image signal representing the ultrasonic image based on the averaged RF data; and a display unit for displaying the ultrasonic image based on the image signal generated by the image signal generating means.

Further, an ultrasonic imaging method according to the present invention includes the steps of: (a) supplying, when sequentially transmitting ultrasonic beams along plural different transmission lines from an ultrasonic probe including plural ultrasonic transducers, drive signals to the plural ultrasonic transducers so as to form an ultrasonic beam having a beam width that covers adjacent two or more transmission lines; (b) performing phase-matching processing and addition processing on reception signals obtained by the plural ultrasonic transducers receiving the ultrasonic echoes to generate RF (radio frequency) data in which focuses of ultrasonic echoes are narrowed in positions corresponding to respective pixels of an ultrasonic image to be displayed; (c) storing the RF data generated at step (b) into an RF data storage unit with respect to a plural number of transmissions of the ultrasonic beams; (d) loading the RF data from the RF data storage unit, averaging the RF data for the plural number of transmissions of the ultrasonic beams, and generating an image signal representing the ultrasonic image based on the averaged RF data; and (e) displaying the ultrasonic image based on the image signal generated at step (d).

According to the present invention, an ultrasonic beam having a beam width that covers adjacent two or more transmission lines is formed, the RF data in which focuses of ultrasonic echoes are narrowed in positions corresponding to respective pixels of an ultrasonic image to be displayed is generated, and the RF data for a plural number of transmissions of ultrasonic beams are averaged, and thereby, the image quality can be improved without increasing the number of channels of ultrasonic transducers, alternatively, the number of channels of ultrasonic transducers can be reduced while good image quality is maintained.

### BRIEF DESCRIPTION OF THE DRAWINGS

FIG. 1 is a block diagram showing a configuration of an ultrasonic diagnostic apparatus according to one embodiment of the present invention;

FIG. 2 shows a condition set in a simulation for obtaining an ultrasonic beam shape;

FIG. 3 shows an ultrasonic beam shape obtained by the simulation;

FIGS. 4A-4C show arrangements of plural ultrasonic transducers;

FIG. 5 shows ultrasonic beam shapes obtained by the simulation based on the arrangements of plural ultrasonic transducers shown in FIGS. 4A-4C;

FIG. 6 shows changes of ultrasonic beam shapes due to apodization for reference;

FIG. 7 shows a Hamming window used for calculation of the ultrasonic beam shapes shown in FIG. 6;

FIG. 8 shows directivity when the width of the ultrasonic transducer is doubled in comparison to a conventional example;

FIG. 9 shows a configuration example of a drive signal generating unit shown in FIG. 1; and

FIGS. 10A-10C are diagrams for explanation of the convex-scan system.

### DESCRIPTION OF THE PREFERRED EMBODIMENTS

Hereinafter, embodiments of the present invention will be described in detail with reference to the drawings. The same reference numerals will be assigned to the same component elements and the description thereof will be omitted.

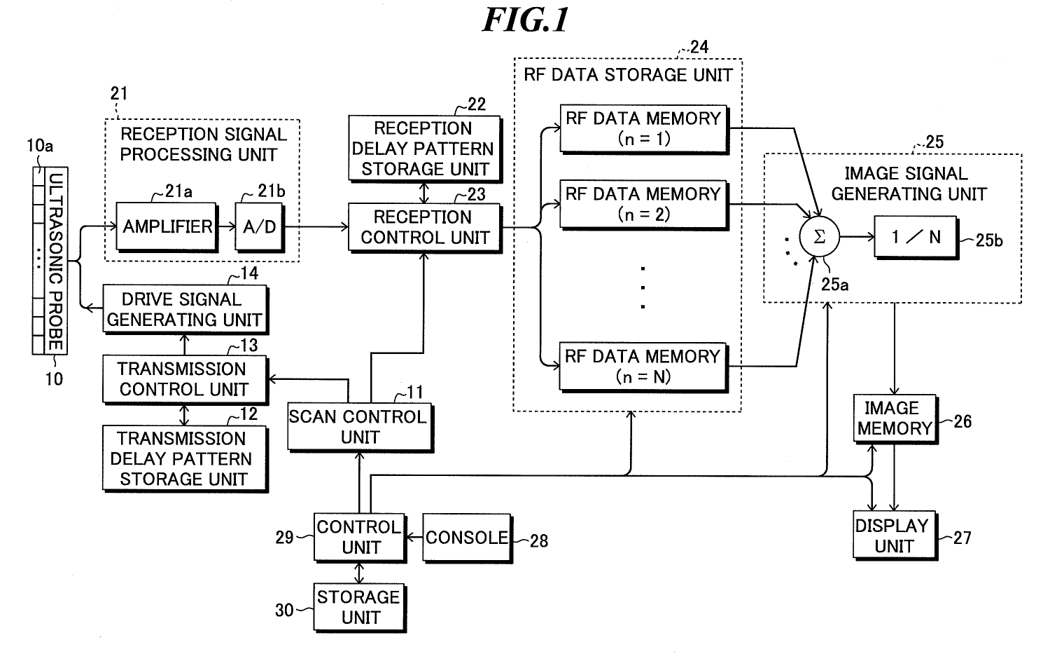

FIG. 1 is a block diagram showing a configuration of an ultrasonic diagnostic apparatus according to one embodiment of the present invention. The ultrasonic diagnostic apparatus includes an ultrasonic probe 10, a scan control unit 11, a transmission delay pattern storage unit 12, a transmission control unit 13, a drive signal generating unit 14, a reception signal processing unit 21, a reception delay pattern storage unit 22, a reception control unit 23, an RF data storage unit 24, an image signal generating unit 25, an image memory 26, a display unit 27, a console 28, a control unit 29, and a storage unit 30.

The ultrasonic probe 10 may be an ultrasonic probe used in contact with an object to be inspected or ultrasonic probe inserted into a body cavity of the object for use. Alternatively, an ultrasonic endoscope may be formed by combining an endoscope for optical observation within the object and an intracavity ultrasonic probe. The ultrasonic probe 10 includes plural ultrasonic transducers 10a forming a one-dimensional or two-dimensional transducer array. These ultrasonic transducers 10a transmit ultrasonic beams based on applied drive signals, and receive propagating ultrasonic echoes and output reception signals.

Each ultrasonic transducer includes a vibrator having electrodes formed on both ends of a material having a piezoelectric property (piezoelectric material) such as a piezoelectric ceramic represented by PZT (Pb(lead)zirconate titanate), a polymeric piezoelectric element represented by PVDF (polyvinylidene difluoride), or the like. When a pulsed or continuous wave voltage is applied to the electrodes of the vibrator, the piezoelectric material expands and contracts. By the expansion and contraction, pulsed or continuous wave ultrasonic waves are generated from the respective vibrators, and an ultrasonic beam is formed by synthesizing these ultrasonic waves. Further, the respective vibrators expand and contract by receiving the propagating ultrasonic waves and generate electric signals. These electric signals are outputted as reception signals of ultrasonic waves.

The scan control unit 11 sequentially sets the transmission directions of ultrasonic beams and the reception directions of ultrasonic echoes. Further, the transmission delay pattern storage unit 12, the transmission control unit 13, and the drive signal generating unit 14 form transmission means. The transmission delay pattern storage unit 12 has stored plural transmission delay patterns used when ultrasonic beams are formed along plural transmission lines. The transmission control unit 13 selects one pattern of the plural delay patterns stored in the transmission delay pattern storage unit 12 according to the transmission lines set in the scan control unit 11, and sets delay times to be provided to drive signals of the plural ultrasonic transducers 10a based on the pattern.

The drive signal generating unit 14 includes plural pulsers corresponding to the plural ultrasonic transducers 10a, for example. The drive signal generating unit 14 adjusts the delay amounts of the drive signals and supplies the drive signals to the ultrasonic probe 10 so that the ultrasonic waves transmitted from the plural ultrasonic transducers 10a may form an ultrasonic beam based on the transmission delay pattern selected by the transmission control unit 13.

In the embodiment, when ultrasonic beams are sequentially transmitted along plural different transmission lines from the ultrasonic probe 10, the drive signal generating unit 14 supplies drive signals to the plural ultrasonic transducers 10a so that the ultrasonic beams have beam widths that cover the adjacent two or more transmission lines.

The reception signal processing unit 21, the reception delay pattern storage unit 22, and the reception control unit 23 form receiving means. The reception signal processing unit 21 includes plural preamplifiers 21a and plural A/D converters 21b corresponding to the plural ultrasonic transducers 10a. The reception signals outputted from the ultrasonic transducers 10a are amplified in the amplifiers 21a and the analog reception signals outputted from the amplifiers 21a are converted into digital reception signals by the A/D converters 21b. The A/D converters 21b output the digital reception signals to the reception control unit 23.

The reception delay pattern storage unit 22 has stored plural reception delay patterns used when reception focus processing is performed on the reception signals outputted from the plural ultrasonic transducers 10a. The reception control unit 23 selects one pattern of the plural reception delay patterns stored in the reception delay pattern storage unit 22 set in the scan control unit 11, and performs phase-matching processing and addition processing by providing delays to the reception signals based on the pattern and adding the signals.

By the reception focus processing, RF (radio frequency) data in which the focuses of the ultrasonic echoes are narrowed in positions corresponding to the respective pixels of an ultrasonic image to be displayed on the display unit 27 are obtained. The RF data represents amplitude and/or phase of the reception signals that have been subjected to reception focus processing, for example.

Further, the reception control unit 23 also serves to convert coordinates of the reception signals obtained by scanning of the object with ultrasonic waves into coordinates of RF data that follows the scan system used in the display unit 27. For example, given that, in the “n”th transmission of ultrasonic beam, a reception signal generated based on ultrasonic echoes at time “t” obtained in the “i”th ultrasonic transducer is S (n,i,t), RF data D (n,x,y) in coordinates (x,y) is expressed by the following equation (1).

$$D ( n , x , y ) = {\sum }_{i} w ( t ) \cdot S \{ n , i , t - τ ( i , x , y ) \} \qquad ( 1 )$$

where W(t) is a function of time “t” and used for weighting, and set to the same with respect to all ultrasonic transducers (transmission directions). Further, τ(i,x,y) is an amount of delay determined depending on the locations of the respective ultrasonic transducers and reception focuses.

As explained above, since the ultrasonic beam has a beam width that covers the adjacent two or more transmission lines, image information on one pixel is included in RF data obtained by at least two ultrasonic beam transmissions. Accordingly, when an ultrasonic beam is repeatedly transmitted at “N” times (N≧2) for imaging of a certain range, RF data D(n,x,y) (n=1, 2,. . . , N) obtained by at least “N” transmissions are stored in an RF data memory of the RF data storage unit 24. The image signal generating unit 25 loads RF data from the RF data storage unit 24, averages RF data for the plural numbers (“N” times) of transmissions with respect to each pixel, and generates an image signal representing an ultrasonic image based on the averaged RF data.

For example, when the RF data represents amplitude of reception signals that have been subjected to reception focus processing, an addition part 25a and a division part 25b of the image signal generating unit 25 perform averaging processing on the RF data according to the following equation (2) to generate an image signal. In this manner, image information on one pixel is generated based on the RF data obtained by ultrasonic beam transmissions at plural times, and thereby, image information is increased and image quality is improved.

$$I ( x , y ) = {\sum }_{n} D ( n , x , y ) / N \qquad ( 2 )$$

In the averaging processing, for example, weighted averages may be obtained according to distances or angles between the transmission lines of ultrasonic beams and the positions of the pixels.

On the other hand, when the RF data represents amplitude and phase of the reception signals that have been subjected to reception focus processing, the image signal generating unit 25 may generate an image signal based on the amplitude of the reception signals represented by the averaged RF data, and generate an image signal representing phase rotation based on the phase of the reception signals represented by the averaged RF data. Furthermore, the image signal generating unit 25 may generate an image signal representing vectors of the reception signals in the respective pixels based on amplitude information and phase information of the reception signals represented by the averaged RF data.

The image signal generated by the image signal generating unit 25 is stored in the image memory 26, and then, supplied to the display unit 30. The display unit 30 includes a display device such as CRT or LCD, for example, and displays a diagnostic image based on the image signal.

The control unit 29 controls the scan control unit 11 and the RF data storage unit 24 to the display unit 30 and so on according to the operation of an operator using the console 28. The above-mentioned scan control unit 11, transmission control unit 13, reception control unit 23, image signal generating unit 25, and control unit 29 can be realized by a CPU and software (programs). The software (programs) is stored in the storage unit 30. As a recording medium in the storage unit 30, not only a built-in hard disk but also a flexible disk, MO, MT, RAM, CD-ROM, DVD-ROM, or the like may be used.

As explained above, according to the embodiment, image quality can be improved without increase in the number of channels of ultrasonic transducers, or the number of channels of ultrasonic transducers can be reduced while good image quality is maintained.

Next, a shape of ultrasonic beam transmitted from plural ultrasonic transducers (hereinafter, also referred to as “elements”) will be explained.

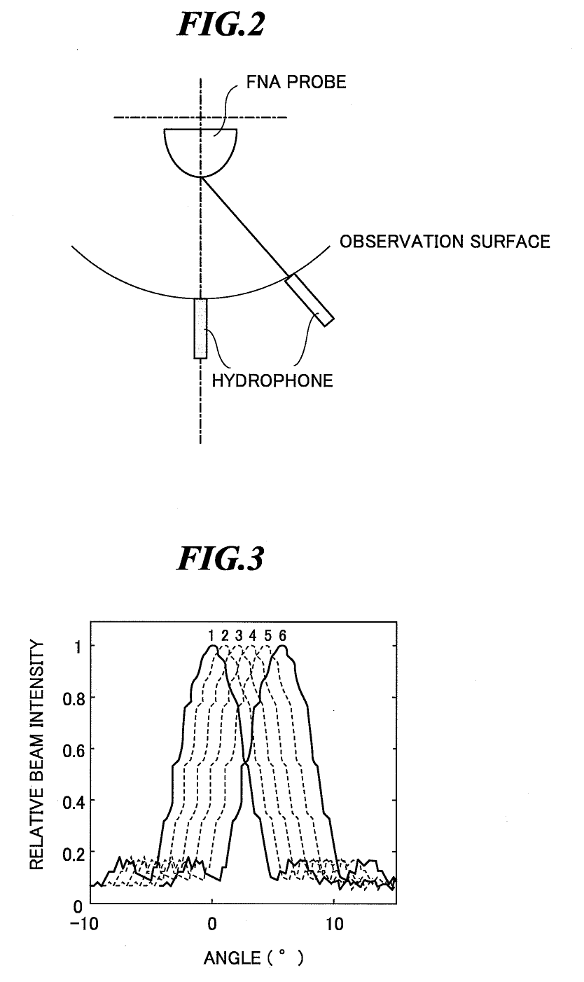

FIG. 2 shows a condition set in a simulation for obtaining an ultrasonic beam shape. In the simulation, a typical FNA probe is assumed. Further, the number of channels of elements used for calculation is 16, the observation point of a hydrophone is on the focus of an acoustic lens, the frequency of ultrasonic waves is 7.5 MHz, and the frequency band of ultrasonic waves is 70%. Note that the band of ultrasonic waves is limited by the waveform of the drive signal and calculated by using a waveform having a frequency band of 40%.

FIG. 3 shows an ultrasonic beam shape obtained by the simulation. In FIG. 3, the horizontal axis indicates an angle from the center line of the FNA probe, and the vertical axis indicates relative intensity of ultrasonic beam (relative beam intensity). When ultrasonic waves having spatially homogeneous intensity are supplied in an imaging range, it is necessary to transmit six ultrasonic beams at the angle from 0° to about 5° as shown by a solid line and a broken line according to the pitch of elements, however, in the embodiment, only the ultrasonic beam shown by the solid line is transmitted in view of the thickness of the ultrasonic beam. That is, the common ultrasonic beam density is excessive for obtaining existing image quality, and, even when the ultrasonic beam density is reduced, the existing image quality can be maintained by effectively utilizing information included in ultrasonic echoes.

In the embodiment, in reception focus processing, image information in the respective pixels on the entire display screen is directly generated from the information included in ultrasonic echoes obtained by one transmission, and thus, image quality deterioration due to interpolation in the digital scan converter (DSC) does not occur. Further, since the information included in ultrasonic echoes obtained by plural transmissions is used for generating image information in one pixel, image quality is not deteriorated even when the density of ultrasonic beam is reduced. Furthermore, since there are plural combinations of positional relationships between transmission points and reception points, speckle suppressive effect is obtained as well.

As shown in FIG. 3, the two ultrasonic beams shown by the solid lines intersect at the relative beam intensity of about 0.5, and accordingly, sufficient beam intensity can be obtained between the ultrasonic beams. When the first ultrasonic beam is transmitted along the first transmission line and then the second ultrasonic beam is transmitted along the second transmission line adjacent to the first transmission line, assuming that the intensity of the first ultrasonic beam in the first transmission line is 100%, it is desirable that the energy of the first ultrasonic beam between the first transmission line and the second transmission line is 30% to 60%. This situation meets the definition that the respective ultrasonic beams have beam widths that cover the adjacent two or more transmission lines.

As described above, when the number of ultrasonic beams is reduced, the number of channels of ultrasonic transducers can be reduced. However, if the number of channels of ultrasonic transducers is simply reduced, a problem of occurrence of a grating lobe arises.

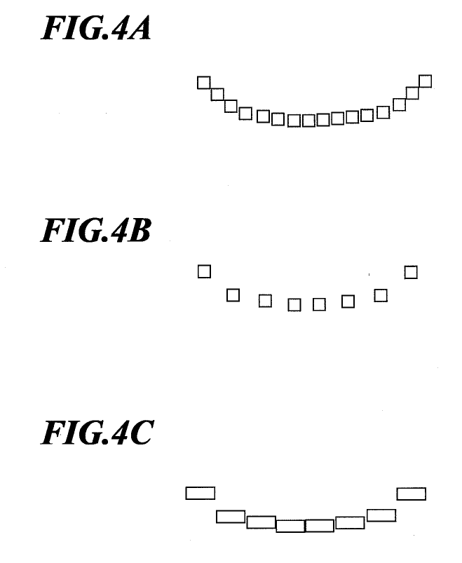

FIGS. 4A-4C show arrangements of plural ultrasonic transducers. In FIG. 4A, 16 elements are arranged as is the case of the simulation. In FIG. 4B, the 16 elements shown in FIG. 4A are thinned to eight. In FIG. 4C, eight elements are arranged with directivity limitations for the respective elements.

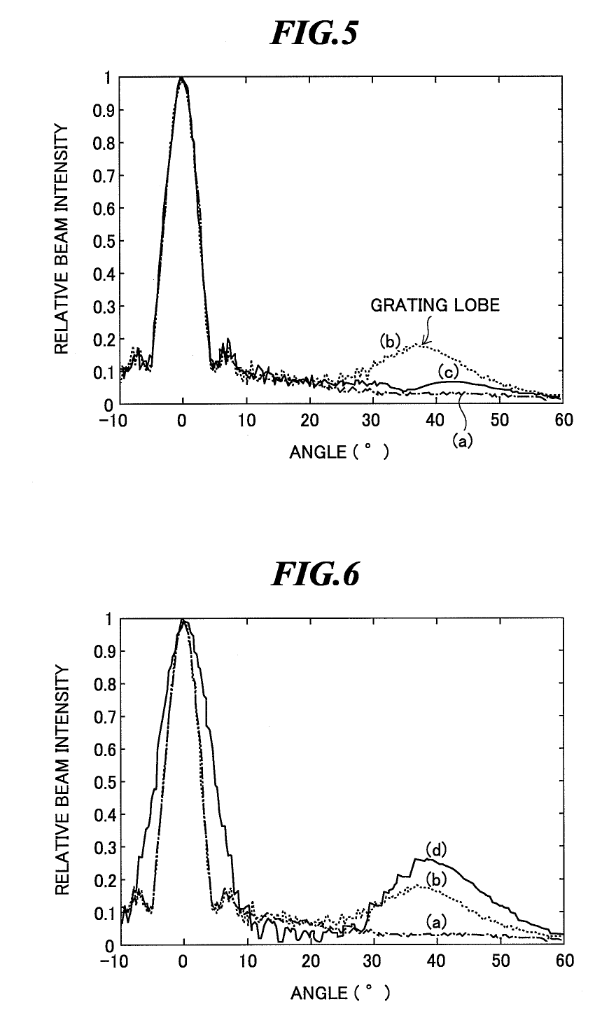

FIG. 5 shows ultrasonic beam shapes obtained by the simulation based on the arrangements of plural ultrasonic transducers shown in FIGS. 4A-4C. In FIG. 5, the horizontal axis indicates the angle (°) from the center line of the FNA probe, and the vertical axis indicates the relative beam intensity. Compared to the case (a) of using 16 elements, in the case (b) of using eight elements, a grating lobe is significant. However, in the case (c) where eight elements are arranged with directivity limitations for the respective elements, the grating lobe is reduced.

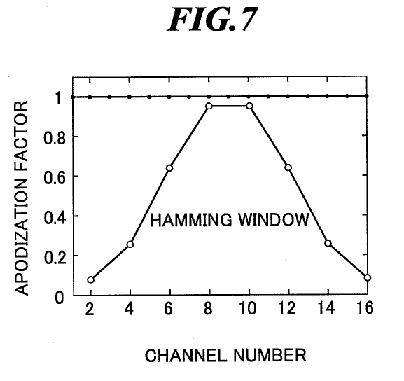

FIG. 6 shows changes of ultrasonic beam shapes due to apodization for reference. Here, (a) and (b) in FIG. 6 are the same as (a) and (b) in FIG. 5, and (d) in FIG. 6 is obtained by apodization using the eight elements shown in FIG. 4B. FIG. 7 shows a Hamming window used for calculation of the ultrasonic beam shapes shown in FIG. 6. In FIG. 7, the horizontal axis indicates a channel number of elements, and the vertical axis indicates an apodization factor. The Hamming window shown in FIG. 7 is formed by changing the apodization factor depending on the element channel. However, as shown in FIG. 6, it was impossible to reduce the grating lobe by apodization.

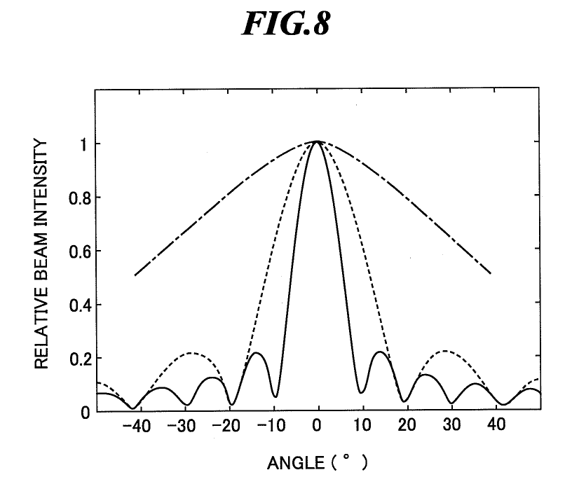

FIG. 8 shows directivity when the width of the ultrasonic transducer is doubled in comparison to a conventional example. In FIG. 8, the horizontal axis indicates the angle (°) from the center line of the FNA probe, and the vertical axis indicates the relative beam intensity. In the conventional example, the directivity of a single ultrasonic transducer (element) is shown by a dashed-dotted line. The directivity shown by a broken line is obtained by increasing the element pitch and increasing the lateral width of the element, however, when an ultrasonic beam is formed by plural elements, grating lobes remain. When the directivity is made sufficiently sharp as shown by a solid line, the grating lobes in the ultrasonic beam are resolved. Therefore, it is desirable that the directivity of single element is sharpened as shown by the solid line. For the purpose, it is effective that the space between plural elements is filled with a hard filling material such as an epoxy resin, or cross talk is allowed to occur in the drive signals supplied to adjacent plural elements.

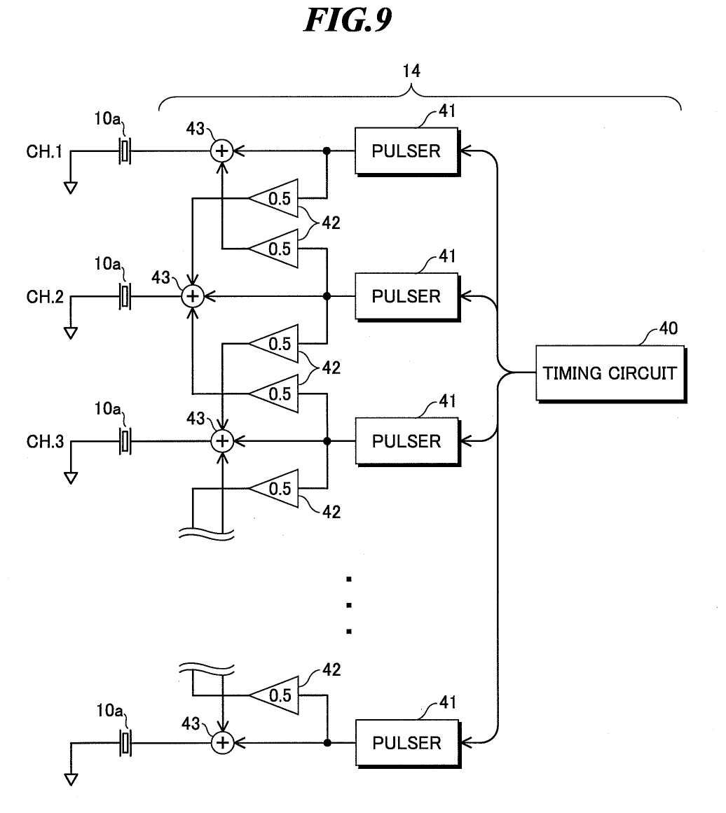

FIG. 9 shows a configuration example of the drive signal generating unit shown in FIG. 1. The drive signal generating unit 14 includes a timing circuit 40 for generating plural timing signals under the control of the transmission control unit 13, plural pulsers 41 for outputting pulsed drive signals according to the respective timings, plural amplifiers 42 with gain of 0.5 for reducing the amplitude of the drive signals outputted from the pulsers 41 to half, and plural adders 43 for adding the drive signals outputted from the amplifiers 42 of adjacent channels to the drive signals of the channels of interest.

In this example, when the plural pulsers 41 supply drive signals to the ultrasonic transducer groups sequentially selected from plural ultrasonic transducers 10a, the transmission line (transmission location and transmission angle) of the ultrasonic beam transmitted from the ultrasonic probe is changed. In this regard, for example, to the ultrasonic transducer 10a of the channel (CH) 2, 100% of the drive signal outputted from the pulser 41 of the channel 2 is applied and 50% of each drive signal outputted from the pulsers 41 of the channels 1 and 3 are applied. Similarly, to the ultrasonic transducers 10a of the channels 1 and 3, 50% of the drive signal outputted from the pulser 41 of the channel 2 is applied. Thereby, the directivity of ultrasonic waves transmitted from the ultrasonic transducers of channels 1-3 becomes sharper than the directivity when ultrasonic waves are transmitted from one ultrasonic transducer.

In this manner, the drive signal generating unit 14 can control the directivity of the ultrasonic transducers 10a independent of the arrangement pitch of the plural ultrasonic transducers 10a. The transmission control unit 13 shown in FIG. 1 may control the drive signal generating unit 14 to cause crosstalk in the drive signals supplied between adjacent plural elements.

Furthermore, in order not to reduce the density of ultrasonic beam so much when reducing the number of channels of ultrasonic transducers, the conventional technique for increasing the density of ultrasonic beam (the number of transmission lines) may be combined to the embodiment. As below, the case of applying such a technique to the convex-scan system will be explained.

The convex-scan system is a modification of the linear-scan system and characterized in that the scan angle can be made larger than that of the linear-scan system. In the convex-scan system, plural elements used at the same time are shifted for moving the ultrasonic beam. Note that the number of transmission lines along which the ultrasonic beams are transmitted becomes smaller than the number of elements because the scan range is limited at ends of the element array.

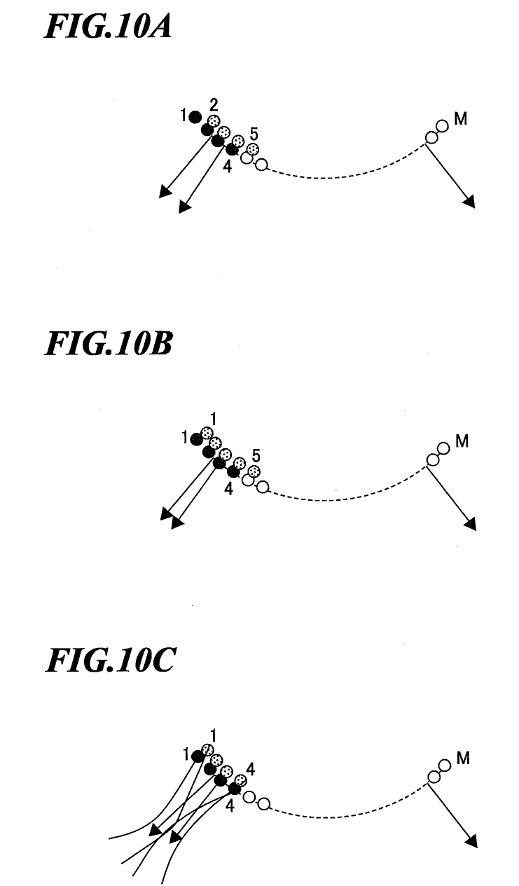

FIGS. 10A-10C are diagrams for explanation of the convex-scan system. As shown in FIG. 10A, “m” (here, m=4) elements are used at the same time in an element array having “M” elements to form one ultrasonic beam (transmission line). The transmission line is moved by shifting the elements used at the same time one by one (black to gray). Accordingly, the number “K” of transmission lines is expressed by the following equation (3).

K=M−m+1 (3)

When the number “m” of elements used for one transmission and reception becomes larger compared to the total number “M” of elements, the number of transmission lines for forming an image becomes smaller. To relax this, processing at ends of the element array is necessary, and, for example, the sector-scan system may be combined at ends of the element array.

FIG. 10B is a diagram for explanation of a half-pitch shift method for increasing the number of transmission lines. As shown in FIG. 10B, an even number (m=4) of elements are used at the same time for an odd-numbered transmission and reception and an odd number (m=5) of elements are used at the same time for an even-numbered transmission and reception, and thus, the amount of movement of the transmission line becomes a half of that in the typical case. Note that the transmission line formed by the ultrasonic waves transmitted from the plural elements is located at the center of an aperture formed by those elements.

FIG. 10C is a diagram for explanation of a small angle sector method for increasing the number of transmission lines. As shown in FIG. 10C, the transmission line is shifted by slightly changing the delay pattern in transmission and reception even using the same elements. Thereby, the number of transmission lines is increased.

1. An ultrasonic imaging apparatus comprising:
an ultrasonic probe including plural ultrasonic transducers for forming an ultrasonic beam according to drive signals to transmit the ultrasonic beam toward an object to be inspected, and receiving ultrasonic echoes generated within the object to generate reception signals;
transmitting means for supplying, when sequentially transmitting ultrasonic beams along plural different transmission lines from said ultrasonic probe, drive signals to said plural ultrasonic transducers so as to form an ultrasonic beam having a beam width that covers adjacent at least two transmission lines;
receiving means for performing phase-matching processing and addition processing on the reception signals obtained by said plural ultrasonic transducers receiving the ultrasonic echoes to generate RF (radio frequency) data in which focuses of ultrasonic echoes are narrowed in positions corresponding to respective pixels of an ultrasonic image to be displayed;
an RF data storage unit for storing the RF data generated by said receiving means with respect to a plural number of transmissions of the ultrasonic beams;
image signal generating means for loading the RF data from said RF data storage unit, averaging the RF data for the plural number of transmissions of the ultrasonic beams, and generating an image signal representing the ultrasonic image based on the averaged RF data; and
a display unit for displaying the ultrasonic image based on the image signal generated by said image signal generating means.

2. The ultrasonic imaging apparatus according to claim 1, wherein said transmitting means controls directivity of said plural ultrasonic transducers independent of an arrangement pitch of said plural ultrasonic transducers.

3. The ultrasonic imaging apparatus according to claim 2, wherein said transmitting means supplies at least part of the drive signal to be supplied to one ultrasonic transducer to at least another ultrasonic transducer adjacent to the one ultrasonic transducer as well.

4. The ultrasonic imaging apparatus according to claim 1, wherein said transmitting means changes a transmission line of the ultrasonic beam to be transmitted from said ultrasonic probe by supplying drive signals to ultrasonic transducer groups sequentially selected from among said plural ultrasonic transducers.

5. The ultrasonic imaging apparatus according to claim 1, wherein the RF data generated by said receiving means represents amplitude and/or phase of the reception signals subjected to phase-matching processing and addition processing.

6. The ultrasonic imaging apparatus according to claim 1, wherein said receiving means converts coordinates of the reception signals obtained by scanning the object with ultrasonic waves into coordinates of RF data that follows a scan system to be used in said display unit.

7. An ultrasonic imaging method comprising the steps of:
(a) supplying, when sequentially transmitting ultrasonic beams along plural different transmission lines from an ultrasonic probe including plural ultrasonic transducers, drive signals to said plural ultrasonic transducers so as to form an ultrasonic beam having a beam width that covers adjacent at least two transmission lines;
(b) performing phase-matching processing and addition processing on reception signals obtained by said plural ultrasonic transducers receiving the ultrasonic echoes to generate RF (radio frequency) data in which focuses of ultrasonic echoes are narrowed in positions corresponding to respective pixels of an ultrasonic image to be displayed;
(c) storing the RF data generated at step (b) into an RF data storage unit with respect to a plural number of transmissions of the ultrasonic beams;
(d) loading the RF data from said RF data storage unit, averaging the RF data for the plural number of transmissions of the ultrasonic beams, and generating an image signal representing the ultrasonic image based on the averaged RF data; and
(e) displaying the ultrasonic image based on the image signal generated at step (d).
